(19) United States
(12) Patent Application Publication
(10) Pub. No.: US 2023/0348698 A1
(43) Pub. Date: Nov. 2, 2023

(54) EMULSION COMPOSITION COMPRISING JELLY COAT GEL PARTICLE

(71) Applicant: AMOREPACIFIC CORPORATION, Seoul (KR)

(72) Inventors: Eunmi KIM, Yongin-si (KR); Seunghan PARK, Yongin-si (KR); Soonae AN, Yongin-si (KR); Eun Soo LEE, Yongin-si (KR); Byungguen CHAE, Yongin-si (KR); Joonho CHOI, Yongin-si (KR)

(73) Assignee: AMOREPACIFIC CORPORATION, Seoul (KR)

(21) Appl. No.: 18/306,070

(22) Filed: Apr. 24, 2023

(30) Foreign Application Priority Data
Apr. 28, 2022 (KR) 10-2022-0052697

(51) Int. Cl. C08L 5/06; C08K 3/16; A61K 8/06; A61K 31/01

(52) U.S. Cl. CPC C08L 5/06; C08K 3/16; A61K 8/06; A61K 31/01; C08K 2003/162; C08L 2203/16

(57) ABSTRACT

The specification relates to an emulsion composition of a formulation that can stably comprise an active substance through a biomimetic structure and can effectively deliver the active substance into the skin. The disclosure uses biocompatible natural vegetable proteins and macromolecules to mimic plant seeds with components and form a morphologically simulated structure of stem cells, so that various efficacious substances can be stably comprised in a high content in the formulation, and high skin absorbability of active substances can be provided. Accordingly, the disclosure can not only solve the safety issues caused by the use of conventional synthetic chemicals, but also enhance the effect of a cosmetic composition or pharmaceutical composition comprising an active substance by stabilizing the active substance in a formulation and effectively absorbing the active substance into the skin.

### CROSS REFERENCE TO RELATED APPLICATION

The present application claims priority to Korean Patent Application No. 10-2022-0052697, filed Apr. 28, 2022, the entire contents of which is incorporated herein for all purposes by this reference.

### BACKGROUND OF THE INVENTION

### Field of the Invention

The disclosure describes an emulsion composition that can stably comprise an active ingredient.

### Description of the Related Art

In the case of a composition comprising two or more thermodynamically immiscible systems of water and oil, a surfactant is required to maintain a stable emulsified emulsion form. As conventional surfactants, synthetic surfactants such as polyglyceryl-based surfactants or polyethylene glycol-based surfactants and various lecithin-based materials close to nature are used. Recently, synthetic polymers in the form of polymeric nanoparticles that can further lower the interface energy have been developed, but safety and environmental issues of organic solvents or the like used dining the manufacturing process have been raised. In addition, in order to increase the long-term stability of the emulsion and prevent precipitation of the hydrophobic active substance collected inside, a large amount of various surfactants are combined or thickeners are used. However, the hydrophobic active substance cannot be comprised in a high content or there is a limitation in its formulation. Further, the use of surfactants and thickeners that are harmful to humans and the environment has still been a problem. For example, there is a limitation that the formulation is limited to a creamy formulation of high hardness or high viscosity with minimized fluidity. Therefore, it is required to develop a new emulsification technology capable of maintaining a stable emulsified formulation in a range of viscosities while being friendly to the human body and the environment.

### SUMMARY OF THE INVENTION

In one aspect, the object to be achieved by the disclosure is to provide an emulsion composition of a formulation that can stably comprise an active substance through a biomimetic structure and can effectively deliver the active substance into the skin.

In one aspect, the disclosure provides an emulsion composition comprising a gel particle comprising an inner film comprising pullulan and an outer jelly coat film comprising an anionic natural polymer and a gelling agent; and an active substance and an amphiphilic natural protein surfactant in the inner film.

In one aspect, the disclosure can stably comprise various active substances in a high content in a formulation by forming a biomimetic structure using biocompatible natural polymers, and can provide high skin absorbability of the active substance. Accordingly, the disclosure can not only solve the safety issues caused by the use of conventional synthetic chemicals, but also enhance the effect of a cosmetic composition or pharmaceutical composition comprising an active substance by stabilizing the active substance in a formulation and effectively absorbing the active substance into the skin.

### BRIEF DESCRIPTION OF THE DRAWINGS

FIG. 1 is a diagram schematically showing gel particles comprised in a composition according to an embodiment of the disclosure.

FIG. 2 is a diagram schematically showing gel particles that are comprised in a composition and further comprises a lipid in an inner film, according to another embodiment of the disclosure.

FIG. 3A shows an image of Example 2 taken with a polarizing microscope.

FIG. 3B shows an image of Example 2 taken with an optical microscope.

FIG. 4A shows an image of Comparative Example 1 taken with a polarizing microscope.

FIG. 4B shows an image of Comparative Example 1 taken with an optical microscope.

FIG. 5 is a diagram showing long-term formulation stability of a composition according to an embodiment of the disclosure by temperature conditions wherein each storage temperature is −20° C., 4° C., 25° C., 30° C., 45° C., cycle conditions of temperature and 60° C. from the left.

FIG. 6A is a diagram showing a formulation of a composition according to an embodiment of the disclosure with a scanning electron microscope immediately after preparation of the composition.

FIG. 6B is a diagram showing a formulation of a composition according to an embodiment of the disclosure with a scanning electron microscope after applying pressure to the composition.

FIG. 7 is a diagram showing a formulation of a composition according to Comparative Example 3 with an optical microscope after applying pressure to the composition.

FIG. 8A is an image of a formulation of Example 3 taken in the XY axis direction using a confocal fluorescence microscope.

FIG. 8B is an image of a formulation of Example 3 taken in the XY axis direction using a confocal fluorescence microscope.

FIG. 8C is an image of a formulation of Example 3 taken in the XY axis direction using a confocal fluorescence microscope.

FIG. 8D is a diagram showing a shape of three-dimensional gel particles formed by combining the images of FIGS. 8A to 8C.

FIG. 8E is a diagram showing a formulation of Example 3 with an optical microscope.

FIG. 8F is a diagram showing a formulation of Example 3 with a polarizing microscope.

FIG. 9A is a diagram showing a formulation of Comparative Example 3 with an optical microscope immediately after preparation of Comparative Example 3.

FIG. 9B is a diagram showing a formulation of Comparative Example 3 with a polarizing microscope immediately after preparation of Comparative Example 3.

FIG. 10A is a diagram showing a formulation of Example 4 with an optical microscope after storage of Comparative Example 4 at 45° C. for 1 month.

FIG. 10B is a diagram showing a formulation of Example 4 with a polarizing microscope after storage of Comparative Example 4 at 45° C. for 1 month.

FIG. 11A is a diagram showing a formulation of Example 4 with an optical microscope.

FIG. 11B is a diagram showing a formulation of Example 4 with a polarizing microscope.

FIG. 12A is a diagram showing a formulation of Example 6 with an optical microscope.

FIG. 12B is a diagram showing a formulation of Example 6 with a polarizing microscope.

FIG. 13A is a diagram showing a formulation of Example 7 with an optical microscope.

FIG. 13B is a diagram showing a formulation of Example 7 with a polarizing microscope.

FIG. 14A is a diagram showing a formulation of Example 8 with an optical microscope.

FIG. 14B is a diagram showing a formulation of Example 8 with a polarizing microscope.

FIG. 15A is a diagram showing a formulation of Comparative Example 5 with an optical microscope immediately after preparation of Comparative Example 5.

FIG. 15B is a diagram showing a formulation of Comparative Example 5 with a polarizing microscope immediately after preparation of Comparative Example 5.

FIG. 16 is a diagram showing measurement and comparison of skin absorbability of a composition (Example 7) according to an embodiment of the disclosure and Comparative Examples 5 and 6.

### DETAILED DESCRIPTION OF THE INVENTION

Embodiments of the disclosure now will be described more fully hereinafter with reference to the accompanying drawings. This disclosure may, however, be embodied in many different forms and should not be construed as limited to the exemplary embodiments set forth therein. Rather, these embodiments are provided so that this disclosure will be thorough and complete, and will fully convey the scope of the disclosure to those skilled in the art. In the drawings, the size of each component, such as width, thickness, etc., is exaggerated to clearly express the component. In addition, although only a part of a component is shown in some cases for convenience of description, those skilled in the art will easily understand the rest of the component. In addition, one of ordinary skill in the art may implement the concept of the disclosure in various other forms without departing from the technical concept of the disclosure.

The singular forms used in the present specification comprise the plural forms, unless the context clearly indicates otherwise. In the application, it will be appreciated that terms “comprising”, “including”, “having” or the like are intended to designate the existence of characteristics, numbers, steps, operations, components described in the specification or a combination thereof, and do not exclude a possibility of the existence or addition of one or more other characteristics, numbers, steps, operations, components, or a combination thereof in advance.

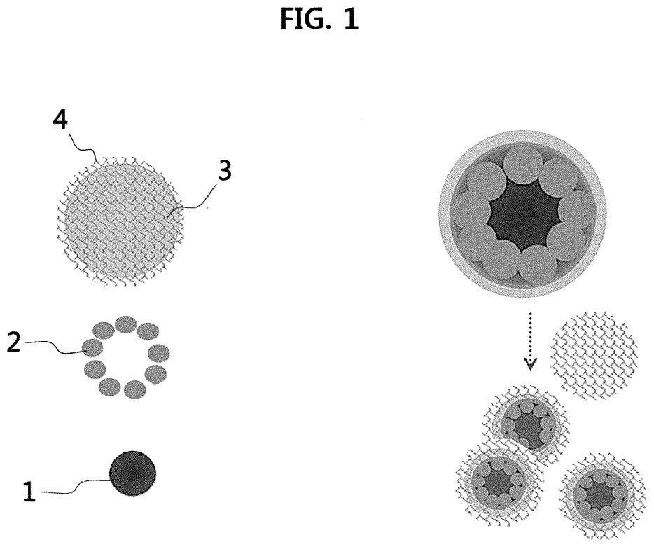

FIG. 1 as accompanied shows an exemplary form of the disclosure. With reference to FIG. 1, one embodiment of the disclosure provides an emulsion composition comprising a gel particle comprising an inner film 3 comprising pullulan and an outer jelly coat film 4 comprising an anionic natural polymer and a gelling agent; and an active substance 1 and an amphiphilic natural protein surfactant 2 in the inner film.

An embodiment of the disclosure relates to a biomimic emulsion formulation for stably collecting active substances inside and effectively delivering them into the skin, which comprises gel particles that further comprising an outer jelly coat film comprising an anionic natural polymer gelled by adding a gelling agent to an anionic natural polymer, that is, gelled anionic natural polymer, on the outermost part of the inner film comprising pullulan. The outer jelly coat film comprised in an embodiment of the disclosure means a film that is flexible and elastic like jelly. For example, it may mean a film in the form of a semi-solid or quasi-solid gel. According to one embodiment, the gel particles may be comprised in the composition in a form separated from each other through the properties of the outer jelly coat film. In addition, since in one embodiment of the disclosure the inner film is coated with the outer jelly coat film, squeezing due to a certain pressure is prevented even when an active substance having fluidity such as oil and an insoluble/hydrophobic active substance are combined or comprised alone, so that the formulation can be stably maintained. In addition, it is possible to add the flexibility to the gel particles that function as a carrier of the active substance when the composition is absorbed into the skin.

In one embodiment, the anionic natural polymer comprised in the outer jelly coat film may be a vegetable polysaccharide. In one embodiment, the anionic natural polymer may be pectin, alginic acid, hyaluronic acid, starch, dextran, carrageenan, cellulose, agarose, agar, a combination thereof, or a salt thereof.

As used herein, “salt” means a salt according to one aspect of the disclosure having the desired activity of a parent compound. Examples of the salt comprise acid addition salt, base addition salt, and amino acid salt. Specifically, there are inorganic acid salts such as hydrochloride, hydrobromide, sulfate, hydroiodide, nitrate and phosphate; organic acid salts such as citrate, oxalate, acetate, formate, propionate, benzoate, trifluoroacetate, maleate, tartrate, methanesulfonate, benzenesulfonate and p-toluenesulfonate; inorganic base salts such as sodium salt, potassium salt, calcium salt, magnesium salt, copper salt, zinc salt, aluminum salt, and ammonium salt; organic base salts such as triethylammonium salt, triethanolammonium salt, pyridinium salt, and diisopropylammonium salt; and amino acid salts such as lysine salt, arginine salt, histidine salt, aspartate, and glutamate.

In one embodiment, the anionic natural polymer may be comprised in an amount of 0.01 to 1% by weight based on the total weight of the composition. In the case that the content of the anionic natural polymer is out of the above range, the outermost film may not be stably formed, and thus the formulation may be separated or the skin absorption rate may be reduced. In one embodiment, the anionic natural polymer may be comprised in an amount of 0.01% by weight or more, 0.02% by weight or more, 0.03% by weight or more, 0.04% by weight or more, 0.05% by weight or more, 0.06% by weight or more, 0.07% by weight or more, 0.08% by weight or more, 0.09% by weight or more, 0.1% by weight or more, 0.2% by weight or more, 0.3% by weight or more, 0.4% by weight or more, 0.5% by weight or more, 0.6% by weight or more, 0.7% by weight or more, 0.8% by weight or more, 0.9% by weight or more, 0.99% by weight or more, 1% by weight or less, 0.9 % by weight or less, 0.8 % by weight or less, 0.7% by weight or less, 0.6% by weight or less, 0.5 by weight or less, 0.4% by weight or less, 0.3% by weight or less, 0.2% by weight or less, 0.1% by weight or less, 0.09% by weight or less, 0.08% by weight or less, 0.0% by weight or less, 0.06% by weight or less, 0.05% by weight or less, 0.04% by weight or less, 0.03% by weight or less, or 0.02% by weight or less, based on the total weight of the composition.

In one embodiment, the gelling agent comprised in the outer jelly coat film may comprise one or more from the group consisting of calcium chloride, calcium carbonate, calcium oxide, and calcium sulfate, but it is not limited thereto as long as it can gel the anionic natural polymer. In one embodiment, the gelling agent may further comprise an organic base salt in addition to calcium chloride, calcium carbonate, or a mixture thereof. In one embodiment, the gelling agent may be comprised in 0.001 to 1% by weight based on the total weight of the composition. In one embodiment, the gelling agent may be comprised in an amount of 0.001% by weight or more, 0.01% by weight or more, 0.02% by weight or more, 0.03% by weight or more. 0.04% by weight or more, 0.05% by weight or more, 0.06% by weight or more, 0.07% by weight or more, 0.08% by weight or more, 0.09% by weight or more, 0.1% by weight or more, 0.2% by weight or more, 0.3% by weight or more, 0.4% by weight or more, 0.5% by weight or more, 0.6% by weight or more, 0.7% by weight or more, 0.8% by weight or more 0.9% by weight or more, or 0.99% by weight or more, 1% by weight or less, 0.9% by weight or less, 0.8% by weight or less, 0.7% by weight or less, 0.6% by weight or less, 0.5% by weight or less, 0.4% by weight or less, 0.3% by weight or less, 0.2% by weight or less, 0.1% by weight or less, 0.09% by weight or less, 0.08% by weight or less, 0.07% by weight or less, 0.06% by weight or less, 0.05% by weight or less, 0.04% by weight or less, 0.03% by weight or less, 0.02% by weight or less, or 0.01% by weight or less, based on the total weight of the composition. Specifically, the weight ratio of the anionic natural polymer and the gelling agent according to one embodiment may be 1:0.0001 to 1. More specifically, the gelling agent is may be comprised in an amount of 0.0001 parts by weight or more, 0.001 parts by weight or more, 0.01 parts by weight or more, 0.02 parts by weight or more, 0.03 parts by weight or more, 0.04 parts by weight or more, 0.05 parts by weight or more, 0.06 parts by weight or more, 0.07 parts by weight or more, 0.08 parts by weight or more, 0.09 parts by weight or more, 0.1 parts by weight or more, 0.2 parts by weight or more, 0.3 parts by weight or more, 0.4 parts by weight or more, 0.5 parts by weight or more, 0.6 parts by weight or more, 0.7 parts by weight or more, 0.8 parts by weight or more, 0.9 parts by weight or more, 1 part by weight or less, 0.9 parts by weight or less, 0.8 parts by weight or less, 0.7 parts by weight or less, 0.6 parts by weight or less, 0.5 parts by weight or less, 0.4 parts by weight or less, 0.3 parts by weight or less, 0.2 parts by weight or less, 0.1 parts by weight or less, 0.09 parts by weight or less, 0.08 parts by weight or less, 0.07 parts by weight or less, 0.06 parts by weight or less, 0.05 parts by weight or less, 0.04 parts by weight or less, 0.03 parts by weight or less, 0.02 parts by weight or less, 0.01 parts by weight or less, or 0.001 parts by weight or less, based on 1 part by weight of the anionic natural polymer. In the case that the weight ratio of the anionic natural polymer and the gelling agent is out of the above range, the outer jelly coat film is not stably formed, and the active substance may be precipitated, resulting in separation of the formulation or reduced skin absorption rate.

In the case that the content of the anionic natural polymer is out of the above range, the outermost film may not be stably formed, and thus the formulation may be separated or the skin absorption rate may be reduced. In one embodiment, the anionic natural polymer may be comprised in an amount of 0.01% by weight or more, 0.02% by weight or more, 0.03% by weight or more, 0.04% by weight or more, 0.05% by weight or more, 0.06% by weight or more, 0.07% by weight or more, 0.08% by weight or more, 0.09% by weight or more, 0.1% by weight or more, 0.2% by weight or more, 0.3% by weight or more, 0.4% by weight or more, 0.5% by weight or more, 0.6% by weight or more, 0.7% by weight or more, 0.8% by weight or more, 0.9% by weight or more, 0.99% by weight or more, 1% by weight or less, 0.9% by weight or less, 0.8% by weight or less, 0.7% by weight or less, 0.6% by weight or less, 0.5% by weight or less, 0.4% by weight or less, 0.3% by weight or less, 0.2% by weight or less, 0.1% by weight or less, 0.09% by weight or less, 0.08% by weight or less, 0.07% by weight or less, 0.06% by weight or less, 0.05% by weight or less, 0.04% by weight or less, 0.03% by weight or less, or 0.02% by weight or less, based on the total weight of the composition.

In one embodiment, the pull-Ulan is an uncharged film-forming polysaccharide and may comprise maltotriose, which is a trisaccharide composed of three glucose molecules linked by α-1,4 glycosidic bonds. In one embodiment, the pullulan is hydrophilic and has filmability and adhesiveness, thereby forming an inner film to primarily provide formulation stability in the composition. In one embodiment, the pullulan absorbs moisture and improves the gelling of the anionic natural polymer having a negative charge, thereby generating the outer jelly coat film in a form different from the thickened form commonly used in the art.

In one embodiment, the pullulan may be comprised in an amount of 0.001 to 1% by weight based on the total weight of the composition. In the case that the pullulan is out of the above range, the outer film may not be effectively formed and the active substance may be precipitated, Specifically, the pullulan may be comprised in an amount of 0.001% by weight or more, 0.01% by weight or more, 0.02% by weight or more, 0.03% by weight or more, 0.04% by weight or more, 0.05% by weight or more, 0.06% by weight or more, 0.07% by weight or more, 0.08% by weight or more, 0.09% by weight or more, 0.1% by weight or more, 0.2% by weight or more, 0.3% by weight or more, 0.4% by weight or more, 0.5% by weight or more, 0.6% by weight or more, 0.7% by weight or more, 0.8% by weight or more, 0.9% by weight or more, 0.99% by weight or more, 1% by weight or less, 0.9% by weight or less, 0.8% by weight or less, 0.7% by weight or less, 0.6% by weight or less, 0.5% by weight or less, 0.4% by weight or less, 0.3% by weight or less, 0.2% by weight or less, 0.1% by weight or less, 0.09% by weight or less, 0.08% by weight or less, 0.07% by weight or less, 0.06% by weight or less, 0.05% by weight or less, 0.04% by weight or less, 0.03% by weight or less, 0.02% by weight or less, 0.01% by weight or less, or 0.005% by weight or less, based on the total weight of the composition.

In one embodiment, the amphiphilic natural protein surfactant may comprise prolamin. The prolamin is a kind of plant storage protein, and is a simple protein comprising a large amount of glutamine and proline. The prolamin has self-assembly hydrophobicity with hydrophobic amino acids such as leucine and isoleucine distributed on its surface. Therefore, the prolamin can effectively collect the active substance by forming a brick-like layered structure surrounding the active substance with a nanorod-like shape. In one embodiment, the prolamin may comprise one or more selected from the group consisting of zein, hordein, secalin, kafirin, gliadin, oryzin, and avenin, but is not limited thereto, and all substances belonging to prolamin may be comprised. Specifically, the zein may be isolated or extracted from corn, the hordein may be isolated or extracted from barley, the secalin may be isolated or extracted from rye, the kafirin may be isolated or extracted from sorghum, gliadin may be isolated or extracted from wheat, the oryzin may be isolated or extracted from rice, and the avenin may be isolated or extracted from oats.

In one embodiment, the amphiphilic natural protein surfactant may be comprised in an amount of 0.0001 to 1% by weight based on the total weight of the composition. The amphiphilic natural protein surfactant serves as a core of the Pickering emulsion through a hydrophobic interaction with the active substance. In the case that it is out of the above range, the gel particles may not be formed or the formulation may be separated. Specifically, the amphiphilic natural protein surfactant may be comprised in an amount of 0.0001% by weight or more, 0.001% by weight or more, 0.01% by weight or more, 0.02% by weight or more, 0.03% by weight or more, 0.04% by weight or more, 0.05% by weight or more, 0.06% by weight or more, 0.07% by weight or more, 0.08% by weight or more, 0.09% by weight or more, 0.1% by weight or more, 0.2% by weight or more, 0.3% by weight or more, 0.4% by weight or more, 0.5% by weight or more, 0.6% by weight or more, 0.7% by weight or more, 0.8% by weight or more, or 0.9% by weight or more, based on the total weight of the composition. In one embodiment, the prolamin may be comprised in an amount of1% by weight or less, 0.9% by weight or less, 0.8% by weight or less, 0.7% by weight or less, 0.6% by weight or less, 0.5% by weight or less, 0.4% by weight or less, 0.3% by weight or less, 0.2% by weight or less, 0.1% by weight or less, 0.09% by weight or less, 0.08% by weight or less, 0.07% by weight or less, 0.06% by weight or less, 0.05% by weight or less, 0.04% by weight or less, 0.03% by weight or less, 0.02% by weight or less, 0.01% by weight or less, or 0.001% by weight or less, based on the total weight of the composition.

In one embodiment, the average particle size of the gel particles may be 1 to 2000 μm. The average particle size means the average of the largest diameters of the particles, and the average particle size means the average size of at least 90% or more of the gel particles distributed in the composition. Specifically, the average particle size may mean the average of the largest diameters of at least 90% or more, 91% or more, 92% or more, 93% or more, 94% or more, 95% or more, 96% or more, 97% or more, 98% or more, or 99% or more of the particles distributed in the composition. Therefore, the composition can stably maintain the structure in which particles having an average particle size of 1 to 2000 μm are evenly distributed for a long period of time without participation, and can function as a delivery system that effectively delivers active substances to the bottom of the stratum contemn. Specifically, the average particle size of the gel particles may be 1 μm or more, 10 μm or more, 20 μm or more, 30 μm or more, 40 μm or more, 50 μm or more, 60 μm or more, 70 μm or more, 80 μm or more, 90 μm or more, 100 μm or more, 200 μm or more, 300 μm or more, 400 μm or more, 500 μm or more, 600 μm or more, 700 μm or more, 800 μm or more, 900 μm or more, 1000 μm or more, 1100 μm or more, 1200 μm or more, 1300 μm or more, 1400 μm or more, 1500 μm or more, 1600 μm or more, 1700 μm or more, 1800 μm or more, 1900 μm or more, 2000 μm or less, 1900 μm or less, 1800 μm or less, 1700 μm or less, 1600 μm or less, 1500 μm or less, 1400 μm or less, 1300 μm or less, 1200 μm or less, 1100 μm or less, 1000 μm or less, 900 μm or less, 800 μm or less, 700 μm or less, 600 μm or less, 500 μm or less, 400 μm or less, 300 μm or less, 200 μm or less, 100 μm or less, 90 μm or less, 80 μm or less, 70 μm or less, 60 μm or less, 50 μm or less, 40 μm or less, 30 μm or less, 20 μm or less, 10 μm or less, or 5 μm or less.

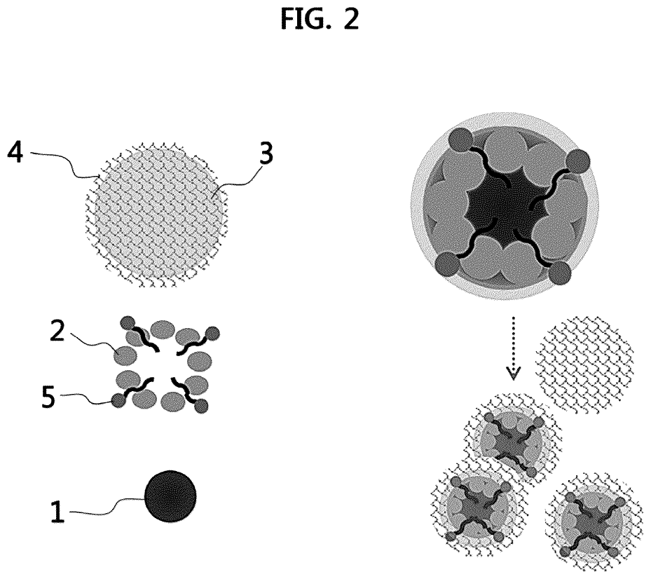

In one embodiment of the disclosure, the inner film 3 may further comprise a lipid 5. FIG. 2 shows air exemplary shape of the gel particle further comprising a lipid in the inner film. The lipid is a component constituting the cell membrane of a plant, and in the disclosure, the lipid may form an interface together with the amphiphilic natural protein surfactant. Specifically, the lipid can form a multi-layered lamellar structure similar to skin lipids together with the amphiphilic natural protein surfactant, thereby enhancing the skin moisture retention effect of the disclosure. The number of carbon atoms in the hydrophobic tail of the lipid is not limited, but, for example, the number of carbon atoms is 6 or more, 7 or more, 8 or more, 9 or more, 10 or more, 11 or more, 12 or more, 13 or more 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or less, 29 or less, 28 or less, 27 or less, 26 or less. 25 or less, 24 or less, 23 or less, 22 or less, 21 or less, 20 or less, 19 or less, 18 or less, 17 or less, 16 or less, 15 or less, 14 or less, 13 or less, 12 or less, 11 or less, 10 or less, 9 or less, 8 or less, or 7 or less. In one embodiment, the lipid may comprise one or more selected from the group consisting of sterol, cholesterol, fatty acid, phytosterol, and ceramide, In one embodiment, the lipid may comprise a C16 or higher alcohol, and the higher alcohol may be, for example, cetyl alcohol, behenyl alcohol, stearyl alcohol, or cetearyl alcohol. In addition, in one embodiment, the lipid can control the size of the gel particles by controlling the number of carbon atoms in the hydrophobic tail, whereby the composition of the disclosure can stably comprise a high content of the active substance. In view of the above, in order to prepare the gel particle comprising the active substance having fluidity in a high content of 3% by weight or more, according to an embodiment, a lipid having 13 to 21 carbon atoms in the hydrophobic tail of the lipid may be further comprised.

In one embodiment, the active substance may be comprised without limitation as long as it is a substance having useful effects on the skin or body. In one embodiment, the active substance itself may be a substance that is difficult for transdermal permeation. In one embodiment, the active substance may be a solid hydrophobic active substance, an oil-soluble liquid active substance, or a mixture thereof. For example, the active substance may comprise one or more selected from an oil selected from the group consisting of squalane, caprylic/capric triglyceride, cetyl ethylhexanoate, 2-octyldodecanol, pentaerythritol tera-2-ethylhexanoate; polyphenol or polyphenol derivative selected from the group consisting of amentoflavone, ellagic acid, apigenin, berginin, diosmetin, univestin, resveratrol, isoflavones and catechin; triterpenoid selected from the group consisting of oleanolic acid, ursolic acid and arjunofic acid; oily fatty acid selected from the group consisting of salicylic acid, alpha lipoic acid, caffeine, tocopherol, DHA-docosahexaenoic acid, eicosapentaenoic acid-EPA and conjugated linolenic acid-CLA; sphingolipid selected from the group consisting of sphingomyelin, ganglioside, cerebroside, ceramide, glycosyl ceramide, lactosyl ceramide, galactosyl ceramide and xylosyl ceramide; oil-soluble vitamin comprising one or more selected from the group consisting of vitamin A, carotene, vitamin E and vitamin K; thymol trimethoxycinnamate; adenosine; saponin and the like.

In one embodiment, the inner film may further comprise a hydrophilic active substance in addition to the solid hydrophobic active substance, the oil-soluble liquid active substance, or a mixture thereof. In this case, the hydrophilic active substance may be adsorbed and comprised in the inner film. The hydrophilic active substance is not limited, but, may comprise, for example, one or more selected from a growth factor such as DNA, RNA, EGF, FGF; mineral such as calcium gluconate, calcium chloride, sodium glycerophosphate, potassium magnesium aspartate, sodium chloride, magnesium gluconate, amino acid; water-soluble vitamin such as vitamin B1, vitamin B2, nicotinamide, pantothenic acid (vitamin B5), vitamin B6, biotin (vitamin B7), and folic acid (vitamin B9); gluconolactone, lactobacillus ferment, enzyme and the like.

In one embodiment, the active substance may be comprised in an amount of 0.1 to 10% by weight based on the total weight of the composition In the case that the active substance is comprised in less than 0.1% by weight, the desired efficacy of the active substance may not be sufficiently exhibited. In one embodiment, in the case that the active substance is comprised in an amount exceeding 10% by weight, the formation of gel particles may be inhibited. Specifically, the active substance may be comprised in an amount of 0.1% by weight or more, 0.2% by weight or more, 0.3% by weight or more, 0.4% by weight or more, 0.5% by weight or more, 0.6% by weight or more, 0.7% by weight or more, 0.8% by weight or more, 0.9% by weight or more, 1% by weight or more, 2% by weight or more, 3 by weight or more, 4% by weight or more, 5% by weight or more, 6% by weight or more, 7% by weight or more, 8% by weight or more, 9% by weight or more, 9.99% by weight or more, 10 by weight or less, 9% by weight or less, 8% by weight or less, 7% by weight or less, 6% by weight or less, 5% by weight or less, 4% by weight or less, 3% by weight or less, 2% by weight or less, 1% by weight or less, 0.9% by weight or less, 0.8% by weight or less, 0.7 by weight or less, 0.6 by weight or less, 0.5% by weight or less, 0.4 by weight or less, 0.3% by weight or less, or 0.2 by weight or less, based on the total weight of the composition.

In one embodiment, the composition may further comprise a thickener. In one embodiment, there is no limitation to the thickener as long as it is a nonionic thickener. In one embodiment, the thickener may comprise a natural or synthetic carbomer, a polyacrylic-based thickener, and the like, and examples thereof comprise cellulose gum and hydroxyethyl cellulose.

An embodiment of the disclosure may provide a method for preparing the composition. In one embodiment, the method may comprise the steps of preparing an oil-phase part by adding and dissolving an amphiphilic natural protein surfactant and an active substance in an alcohol solvent; preparing an aqueous-phase part by adding and dissolving pullulan and an anionic natural polymer in an aqueous solvent; adding the oil-phase part into the prepared aqueous-phase part and forming a gel particle by adding a gelling agent to the aqueous-phase part and gelling the aqueous-phase part.

In one embodiment, the step of adding and dissolving the amphiphilic natural protein surfactant and the active substance in the alcohol solvent may comprise adding the amphiphilic natural protein surfactant to the alcohol solvent, and adding the active substance thereto to disperse the active substance. The amphiphilic natural protein surfactant is a simple protein soluble in 60 to 90% alcohol, which is soluble in dilute alcohol but insoluble in water or anhydrous alcohol solution. Therefore, in one embodiment, the amphiphilic natural protein surfactant added to the alcohol solvent may be the surfactant that is dissolved in 60 to 90% alcohol. In addition, in one embodiment, the alcohol solvent into which the active substance is added may be 70 to 95% alcohol. As described above, when the amphiphilic natural protein surfactant and the active substance are added to the alcohol, a structure in which the amphiphilic natural protein surfactant collects the active substance due to the hydrophobic interaction between the active substance and the amphiphilic natural protein surfactant may be formed. In one embodiment, the alcohol solvent may comprise a polyhydric alcohol, but is not limited thereto. For example, the polyhydric alcohol may be a polyol, and specifically, the polyol may comprise one or more selected from the group consisting of glycerin, 1,2-hexanediol, polyethylene glycol, polypropylene, glycol, dipropylene glycol, propylene glycol, butylene glycol, polyglycerin-3, propanediol, sorbitol, erythritol, xylitol, maltitol, ethylhexanediol, PEG/PPG/polybutylene glycol-8/5/3 glycerin, ethylhexanediol, and pentylene glycol.

In one embodiment, in the step of adding and dissolving the amphiphilic natural protein surfactant and the active substance in the alcohol solvent, the dissolution temperature, may be 65 to 95° C.

In one embodiment, after the step of preparing the oil-phase part, the step of adding the lipid to the prepared oil-phase part may be further comprised.

In one embodiment, in the step of preparing the water-phase part by adding and dissolving the pullulan and the anionic natural polymer in an aqueous solvent, the aqueous solvent may comprise water.

In one embodiment, in the previous step of adding the gelling agent and gelling to form the gel particles, when the oil-phase part is added to the water-phase part, instantaneous emulsification is achieved by electrostatic attraction between the amphiphilic natural protein surfactant and the anionic natural polymer. Here, the step of adding the gelling agent and gelling to form gel particles may comprise forming the gel particles by adding the gelling agent and gelling the anionic natural polymer at the outermost part of the inner film to form the outer jelly coat film. Here, in one embodiment, the gelling agent may be liquid. For example, the gelling agent may be an aqueous calcium chloride solution, an aqueous calcium carbonate solution, or a mixture thereof.

In one embodiment, the disclosure may provide a composition for external application for skin.

The composition according to one embodiment of the disclosure may be a cosmetic composition.

In one embodiment, the cosmetic composition according to the disclosure may be formulated by comprising a cosmetically or dermatologically acceptable medium or substrate. Examples of the formulations for topical application may comprise a solution, a gel, a solid, an anhydrous paste, an oil-in-water emulsion, a suspension, a microemulsion, a microcapsule, a microgranule, an ionic (liposome) or non-ionic vesicular dispersion, a film, a cream, a skin lotion, a lotion, a powder, an ointment, a spray or a conceal stick. The composition can be formulated according the methods commonly employed in the related art.

In one embodiment, the cosmetic composition according to the disclosure may further comprise, in addition to the above-described active substance, other ingredients that may preferably provide a synergic effect to the main effect within a range not negatively affecting the main effect. The ingredients other than the active substance of the disclosure may be selected by those skilled in the art without difficulty depending on the formulation type or purpose of use of the cosmetic composition. In one embodiment, the cosmetic composition of the disclosure may also comprise, in addition to the above ingredients, other ingredients commonly mixed in a cosmetic composition as needed. Examples may comprise a humectant, an emollient, an organic or inorganic pigment, an organic particle, a UV absorbent, an antiseptic, a sterilizer, an antioxidant, a plant extract, a pH control agent, an alcohol, a coloring agent, a fragrance, a blood circulation stimulant, a cooling agent, an antiperspirant, purified water, etc. However, the other ingredients that may be comprised in the cosmetic composition of the disclosure are not limited thereto and the amount thereof may be determined within a range not negatively affecting the purpose and effect of the disclosure.

The composition according to embodiments of the disclosure may be a pharmaceutical composition. The pharmaceutical composition may further comprise a pharmaceutical adjuvant such as an antiseptic, a stabilizer, a hydrating agent, an emulsification accelerator, a salt and/or buffer for control of osmotic pressure, etc. and other therapeutically useful substances. In one embodiment, the pharmaceutical composition may be a formulation for parenteral administration. The formulation for parenteral administration may be a formulation for rectal, topical, subcutaneous or transdermal administration. For example, the formulation may be an injection, a medicinal drip, an ointment, a lotion, a gel, a cream, a spray, a suspension, an emulsion, a suppository, a patch, etc., although not being limited thereto.

In one embodiment, the dosage of the pharmaceutical composition will vary depending on the age, sex and body weight of a subject to be treated, the particular disease or pathological condition to be treated, the severity of the disease or pathological condition, administration route and discretion of a prescriber. The determination of the dosage considering these factors is within the level of those skilled in the art.

In one embodiment, when applied to the skin, the administration dose of the composition may be 1 mg/kg/day to 100 g/kg/day. In one embodiment, the dosage of the composition will vary depending on the age, sex and body weight of a subject to be treated, the particular disease or pathological condition to be treated, the severity of the disease or pathological condition administration route, or the like, and the determination of the dosage considering these factors is within the level of those skilled in the art. For example, the dosage may be 1 mg/kg/day or more, 10 mg/kg/day or more, 100 mg/kg/day or more, 1 g/kg/day or more, 5 g/kg/day or more, 10 g/kg/day or more, 20 g/kg/day or more, 30 g/kg/day or more, 40 g/kg/day or more, 50 g/kg/day or more, 60 g/kg/day or more, 70 g/kg/day or more, 80 g/kg/day or more, 90 g/kg/day or more, 99 g/kg/day or more, 100 g/kg/day or less, 90 g/kg/day or less, 80 g/kg/day or less, 70 g/kg/day or less, 60 g/kg/day or less, 50 g/kg/day or less, 40 g/kg/day or less, 30 g/kg/day or less, 20 g/kg/day or less, 10 g/kg/day or less, 1 g/kg/day or less, 100 mg/kg/day or less, 10 mg/kg/day or less, or 5 mg/kg/day or less, but the dosage does not limit the scope of the disclosure by any means.

Hereinafter, the present disclosure will be described in detail through examples, comparative examples and experimental examples. However, the following examples are for illustrative purposes only and it will be obvious to those of ordinary skill in the art that the scope of the disclosure is not limited by the examples, comparative examples and experimental examples.

### Preparation Example

A composition according to an embodiment of the disclosure was prepared according to the following method with the composition shown in Table 1 below.

Specifically, the composition is prepared by the steps of preparing an oil-phase part by adding and dissolving an amphiphilic natural protein surfactant and an active substance in an alcohol solvent; preparing an aqueous-phase part by adding and dissolving pullulan and an anionic natural polymer in an aqueous solvent; adding the oil-phase part into the prepared aqueous-phase part; and forming a gel particle by adding a gelling agent to the aqueous-phase part and gelling the aqueous-phase part.

TABLE 1

| | | | | | Comparative | Comparative | Comparative | Comparative |
|---|---|---|---|---|---|---|---|---|
| | INCI name | Example 1 | Example 2 | Example 3 | Example 1 | Example 2 | Example 3 | Example 4 |
| Aqueous | Water | To 100 | To 100 | To 100 | To 100 | To 100 | To 100 | To 100 |
| solvent | | | | | | | | |
| Alcohol | Glycerin | 5 | 5 | 5 | 5 | 5 | 5 | 5 |
| solvent | 1,2-hexanediol | 1 | 1 | 1 | 1 | 1 | 1 | 1 |
| Lipid | Stearyl alcohol | 0.5 | 0.5 | 0.5 | 0.5 | 0.5 | 0.5 | 0.5 |
| Active | Squalane | 5 | 5 | 5 | 5 | 5 | 5 | 5 |
| substance | | | | | | | | |
| Amphiphilic | Zein | 0.1 | 0.05 | 0.05 | 0.5 | 0.0005 | — | 0.05 |
| natural | | | | | | | | |
| protein | | | | | | | | |
| stabilizer | | | | | | | | |
| Pullulan | Pullulan | 0.4 | 0.4 | 0.4 | 1.5 | 1.5 | 0.4 | — |
| Active | Hydrolyzed | — | 0.1 | 0.1 | — | — | — | 0.1 |
| substance | ginseng | | | | | | | |
| | saponin(BioGF1K) | | | | | | | |
| Active | Oleanolic acid | — | — | 0.05 | — | — | — | — |
| substance | | | | | | | | |
| Anionic | Pectin | 0.1 | 0.1 | 0.1 | 0.1 | 1.5 | 0.3 | 0.3 |
| natural | | | | | | | | |
| polymer | | | | | | | | |
| Gelling | Calcium chloride | 0.03 | 0.03 | 0.03 | 0.01 | 0.1 | 0.03 | 0.03 |
| agent | | | | | | | | |
| Thickener | Hydroxyethyl | 5 | 5 | 5 | 5 | 5 | 5 | 5 |
| | cellulose | | | | | | | |
| Total(wt %) | 100 | 100 | 100 | 100 | 100 | 100 | 100 | 100 |

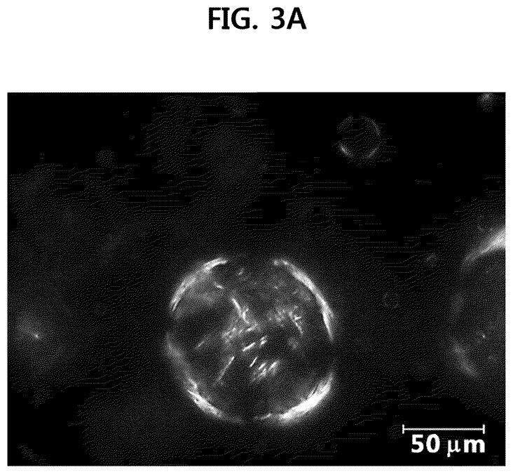

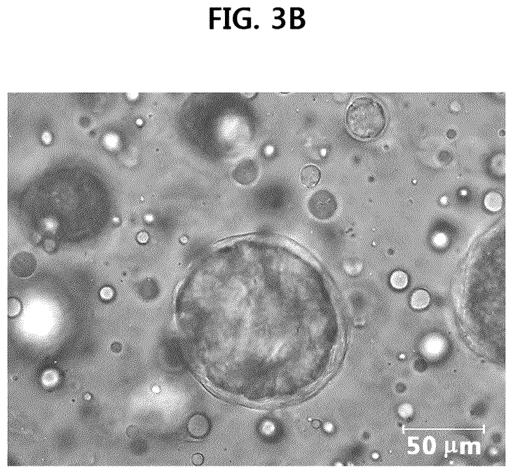

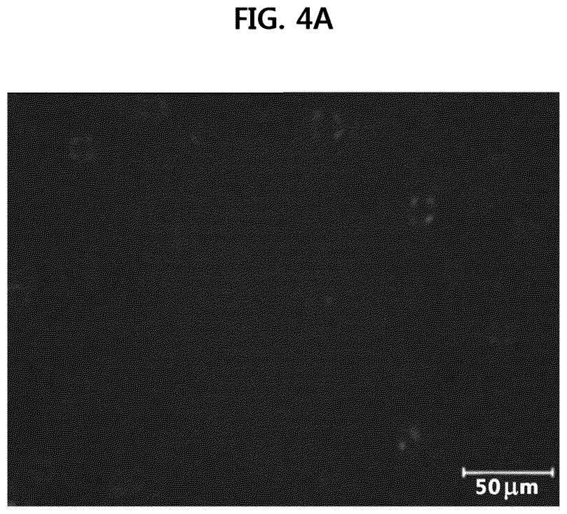

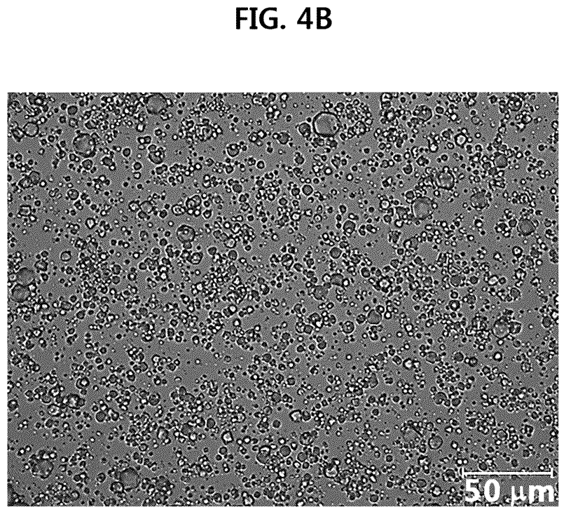

As a result, in Examples 1 and 2, the gel particles comprising the outer jelly coat film were formed. FIGS. 3A and 3B show images of Example 2 taken with a polarizing microscope (FIG. 3A) and an optical microscope (FIG. 3B). In the case of Example 2, the formation of the jelly coat film was confirmed with the polarizing image and the optical image. On the other hand, Comparative Examples 1 and 2 did not form the outer jelly coat film, and Comparative Example 2 did not form an emulsion, resulting in separation of the formulation. FIGS. 4A and 4B show images of Comparative Example 1 taken with a polarizing microscope (FIG. 4A) and an optical microscope (FIG. 4B), and since the outer jelly coat film was not formed, no gel particles were observed in the polarization image.

Here, the polarizing microscope and optical microscope used were Nikkon ECLIPSE 80i (Olympus), respectively, and the same products were used in the experiments below.

### Experimental Example

In order to confirm the long-term formulation stability of the disclosure, the following experiments were conducted.

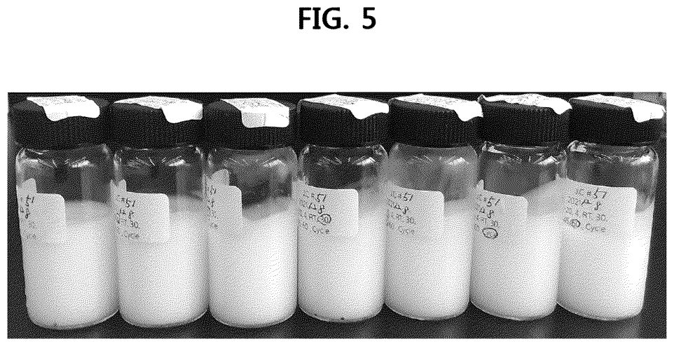

The composition of Example 2 was stored for 1 month at −20° C., 4° C., 25° C., 30° C., 45° C., 60° C., or a cycle condition of temperatures (cycled at −15° C. to 45° C. for 12 hours). Afterwards, changes in whether the active substance was precipitated and whether the formulation was separated were confirmed. As a result, as shown in FIG. 5, it was confirmed that in Example 2 of the disclosure, the formulation was stably maintained without precipitation or discoloration of the active substance even after storage for 1 month under each constant temperature storage condition.

### Experimental Example

In order to confirm whether the precipitation of the active substance in the inner phase is prevented when a certain external pressure is applied to the composition of the disclosure, the following experiment was conducted.

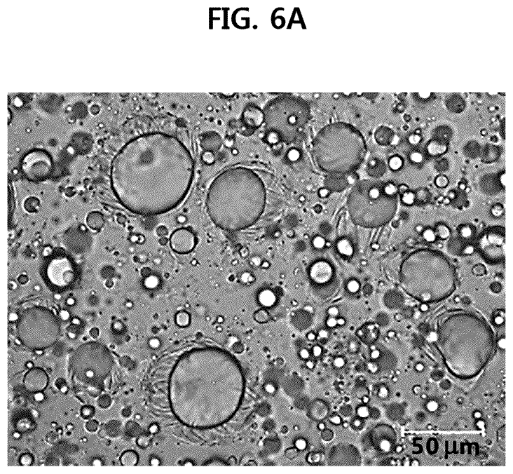

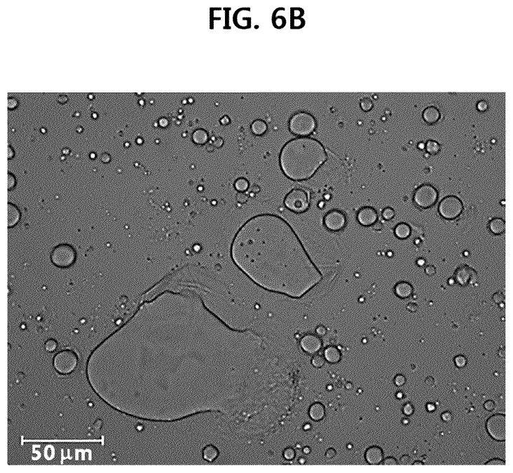

First, the formulation immediately after preparing the composition of Example 1 was confirmed with an optical microscope, which was shown in FIG. 6A. The composition with the formulation was taken out, placed on a 100 μg slide glass, covered with a cover glass, and pressure was applied with a weight of approximately 5 N (500 g weight) for 10 seconds, and then the formulation of the composition was confirmed again with an optical microscope, which was shown in FIG. 6B, respectively.

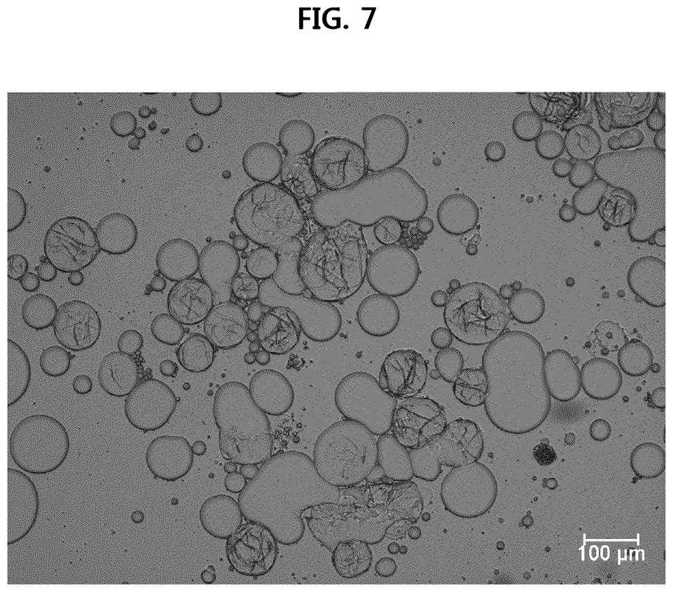

In addition, as a comparative example, according to the composition described in Table 1 above, the gel particles comprising only an outer film obtained by gelling pectin with CaCl₂ without comprising the amphiphilic natural protein surfactant were prepared (Comparative Example 3). After applying the pressure by the above method, the composition with the formulation was confirmed and shown in FIG. 7. As a result, in Example 1 of the disclosure, it was confirmed that the squalane oil, which is an active substance, was prevented from being squeezed by the inner film and the outer jelly coat film, thereby preventing precipitation of the active substance. In addition, it was confirmed that the gel particles of Example 1 of the disclosure were dispersed in the formulation without aggregation due to the negative charge and elasticity of the outer jelly coat film. On the other hand, in Comparative Example 3 comprising only the outer jelly coat film, the formulation stability was poor, so that the oil escaped, and as a result, the oil-phase was completely separated from the aqueous-phase.

### Experimental Example

In order to confirm the capture stability of the active substance of the composition according to the disclosure, the following experiment was conducted.

In one embodiment of the disclosure, the composition (Example 3) was prepared in the same manner as in Example 1 above, except that 0.0001 to 0.1% by weight of oleanolic acid and 0.00001 to 0.5% by weight of saponin (product name: BioGf1K, manufacturer: HYUNDAI BIOLAND Co., Ltd) as the active substances were comprised based on the total weight of the composition, Here, in order to confirm whether the active substance was precipitated, the hydrophobic dye. Nile red (Sigma), was further comprised in the active substance collected in the inner film to stain the inner film, and hyaluronic acid tagged with the hydrophilic FIT-C dye (NAT-167 FITC-labelled Hyaluronic Acid, Matexcel) was further comprised in the inner film to stain the inner film.

After storing the prepared Example 3 at room temperature (25° C.) and high temperature (45° C.) for 1 month, respectively, confocal fluorescence images (product name: LSM 980 NLO, Confocal microscope, manufacturer: ZEISS) was taken to confirm Whether the active substance was precipitated,

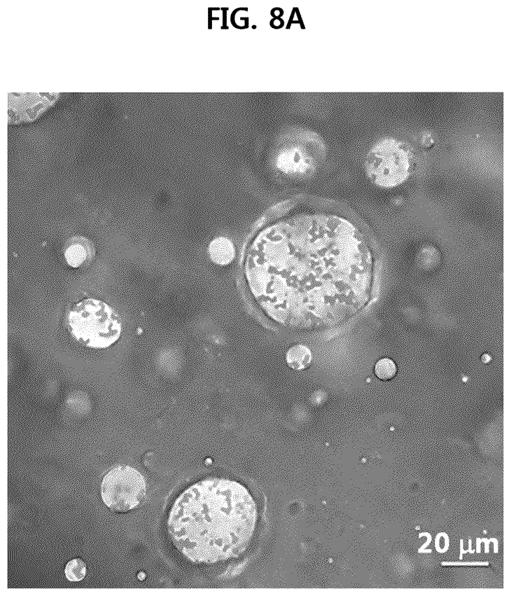

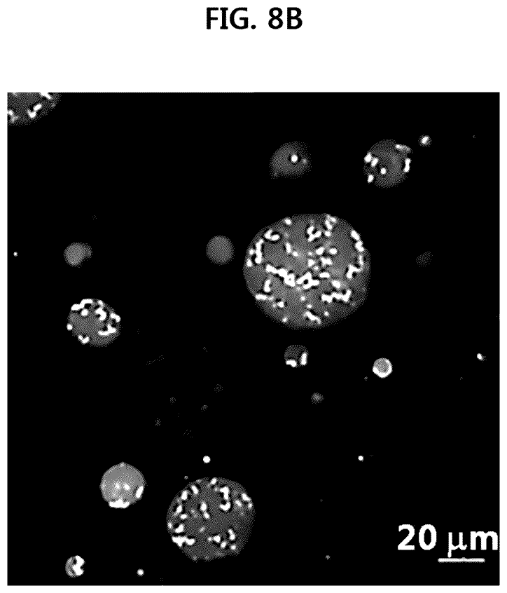

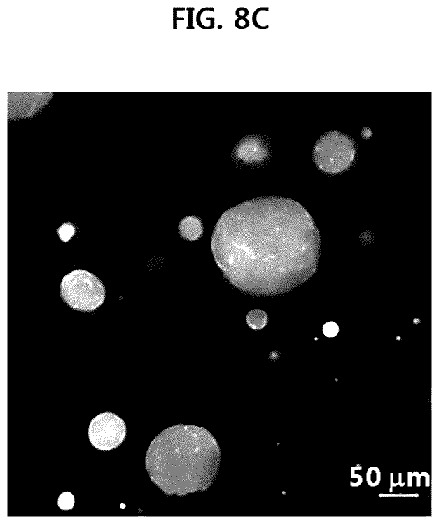

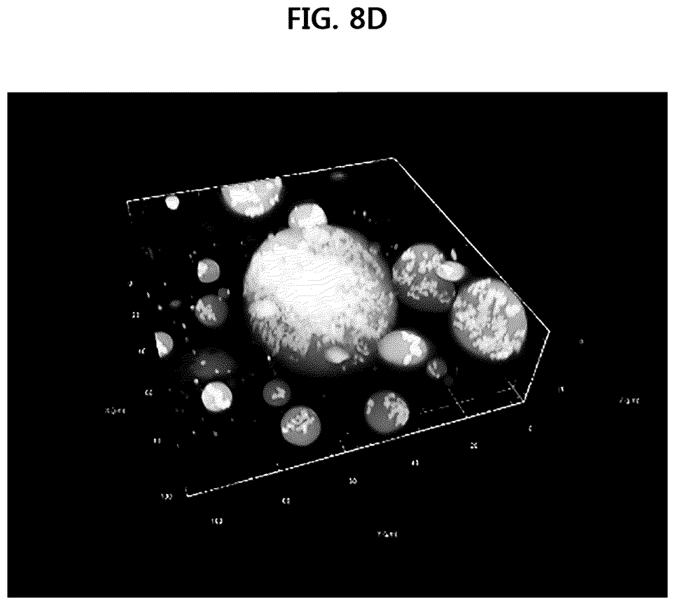

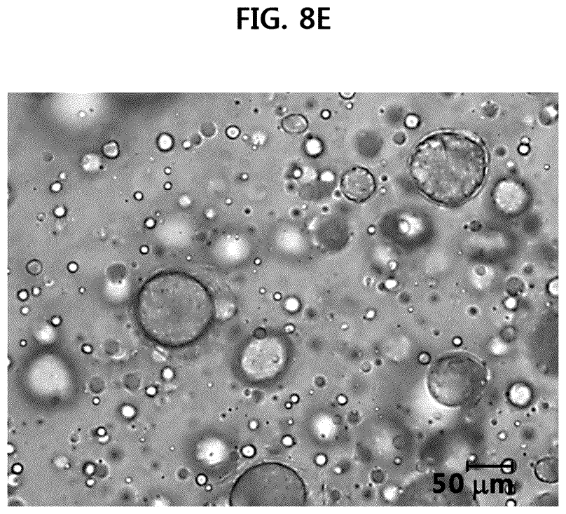

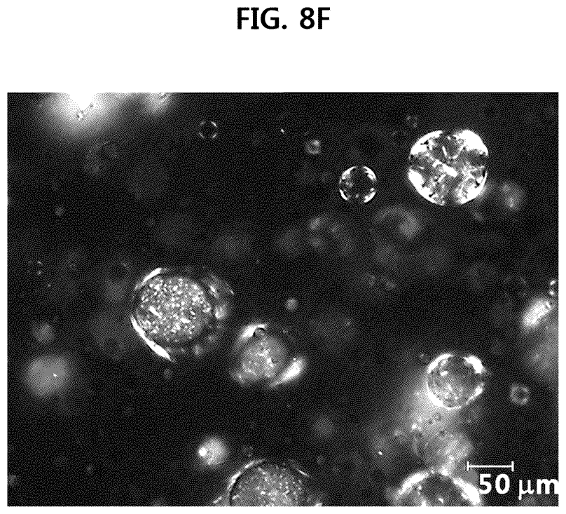

FIGS. 8A to 8C are two-dimensional images of the gel particles, and FIG. 8D shows a three-dimensional image of the shape of the gel particles by combining the two-dimensional images. FIG. 8A shows the distribution form of the hydrophilic/hydrophobic substances in the formulation and the form of the jelly coat, which is the outer film, in a complex manner. FIG. 8B shows the distribution pattern of the hydrophobic substance in the formulation. FIG. 8C shows the distribution pattern of the hydrophilic substance in the formulation. FIG. 8D shows the distribution shape of hydrophilic and hydrophobic substances in the formulation. As shown in the drawings, in Example 3, it can be confirmed that the red core comprising the active substance was well collected in the green inner film, so that the emulsion particles were stably formed without separation. FIGS. 8E and 8F show the formulation of Example 3 immediately after preparation of Example 3 with an optical microscope (FIG. 8E) and a polarizing microscope (FIG. 8F) and the oleanolic acid sparkling inside the emulsified particles was observed. Since the insoluble component such as oleanolic acid is difficult to maintain in a dissolved state in the aqueous-phase, hydrophobic interactions between particles lead to aggregation and precipitation, so that there is a limit to maintaining long-term stability in emulsion formulations. However, according to the examples of the disclosure, it can be confirmed that oleanolic acid was comprised in the inner film of the gel particles comprising the outer jelly coat film and did not precipitate out of the emulsion, and the gel particles did not precipitate. That is, in Example 3 according to an embodiment of the disclosure, even when stored for a long time at room temperature or high temperature, the precipitation of the active substance by the outer jelly coat film was prevented, and the formulation stability was maintained for a long time.

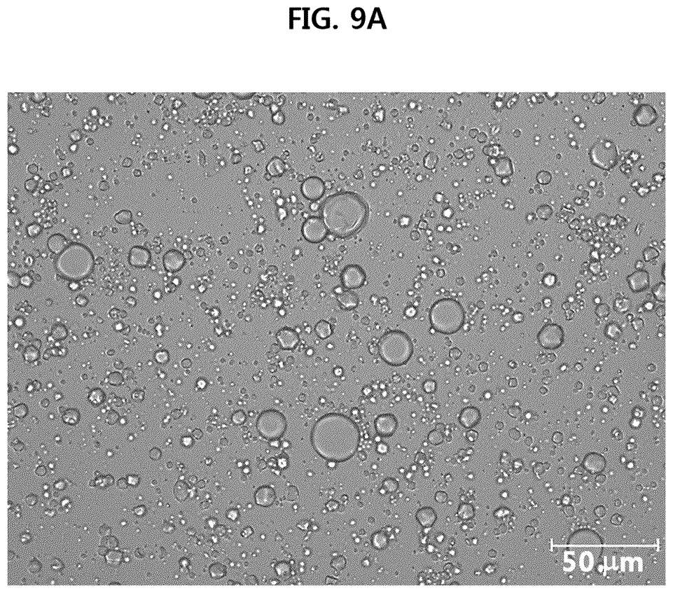

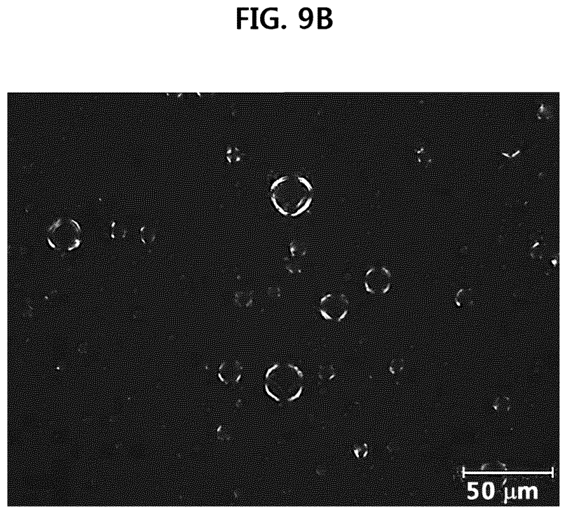

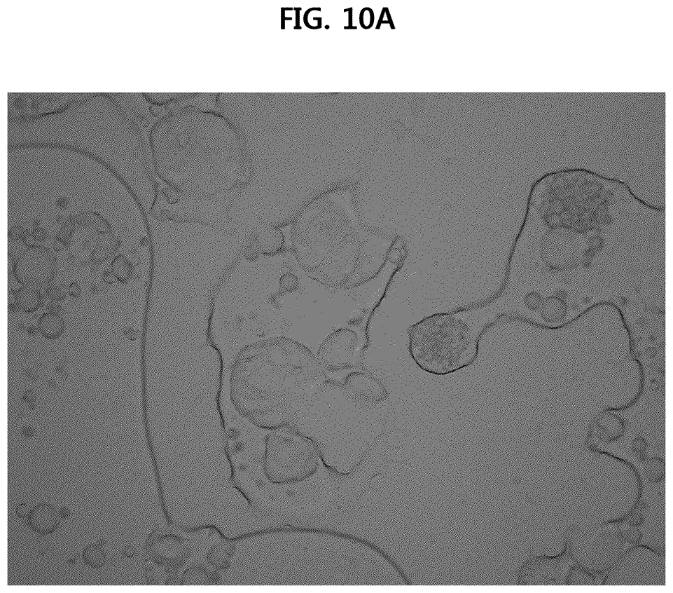

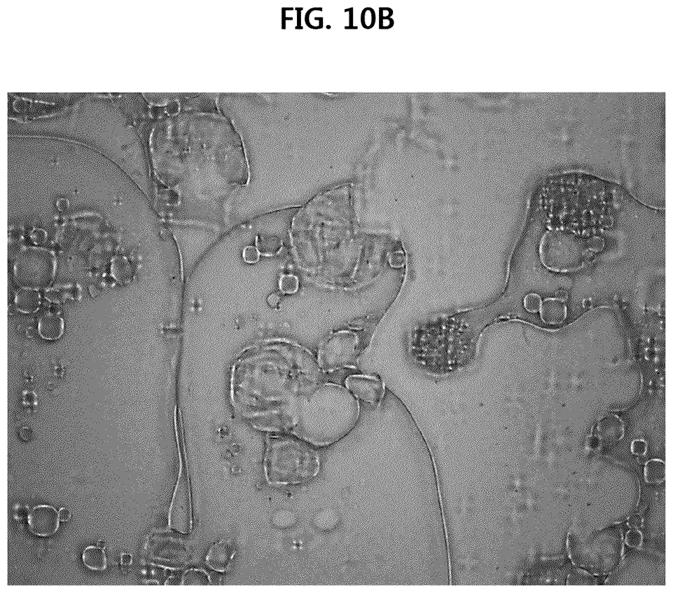

In addition, as a comparative example, immediately after preparing Comparative Example 3 of Table 1, the formulation of Comparative Example 3 at room temperature was confirmed with an optical microscope (FIG. 9A) and a polarizing microscope (FIG. 9B) After Comparative Example 4 was stored at 45° C. for 1 month, the formulation of Comparative Example 4 was confirmed with an optical microscope (FIG. 10A) and a polarizing microscope (FIG. 10B). Here, the scale bars in FIGS. 10A and 10B are the same as those in FIGS. 9A and 9B. As a result, as shown in FIGS. 10A and 10B, Comparative Example 3, which did not comprise the amphiphilic natural protein surfactant, showed precipitation of the active substance when stored at high temperature for a long period of time, unlike Example 3 of the disclosure.

### Experimental Example 4

In one embodiment of the disclosure, Examples 4 to 8 in Table 2 below were prepared in the same manner as in Example 1, except that lipid was further comprised in the inner film.

TABLE 2

| | INCI name | Example 4 | Example 5 | Example 6 | Example 7 | Example 8 |
|---|---|---|---|---|---|---|
| Aqueous | Water | To 100 | To 100 | To 100 | To 100 | To 100 |
| solvent | | | | | | |
| Alcohol | Glycerin | 5 | 5 | 5 | 5 | 5 |
| solvent | 1,2-hexanediol | 1 | 1 | 1 | 1 | 1 |
| Lipid | Lauryl alcohol | — | — | 0.5 | — | — |
| | Behenyl alcohol | — | — | — | — | 0.5 |
| | Stearyl alcohol | — | — | — | 0.5 | — |
| Active | Squalane | 2 | 5 | 5 | 5 | 5 |
| substance | | | | | | |
| Amphiphilic | Zein | 0.05 | 0.05 | 0.05 | 0.05 | 0.05 |
| natural | | | | | | |
| protein | | | | | | |
| stabilizer | | | | | | |
| Pullulan | Pullulan | 0.4 | 0.4 | 0.4 | 0.4 | 0.4 |
| Active | Hydrolyzed ginseng | 0.1 | 0.1 | 0.1 | 0.1 | 0.1 |
| substance | saponin, BioGF1K | | | | | |
| Anionic | Pectin | 0.1 | 0.1 | 0.1 | 0.1 | 0.1 |
| natural | | | | | | |
| polymer | | | | | | |
| Gelling agent | Calcium chloride | 0.03 | 0.03 | 0.03 | 0.03 | 0.03 |
| Thickener | Hydroxyethyl | 5 | 5 | 5 | 5 | 5 |
| | cellulose | | | | | |
| Total(wt %) | 100 | 100 | 100 | 100 | 100 | 100 |

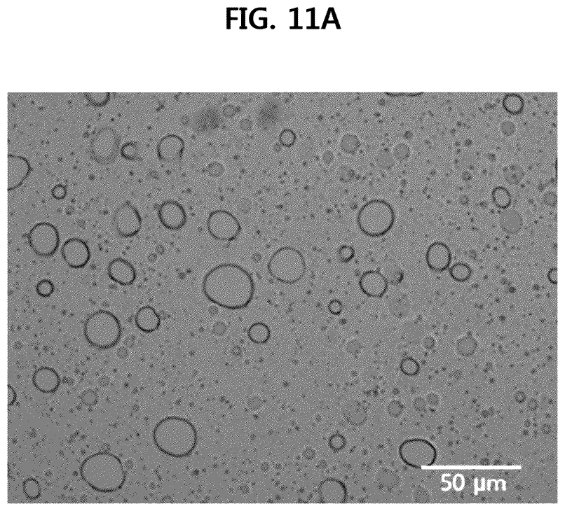

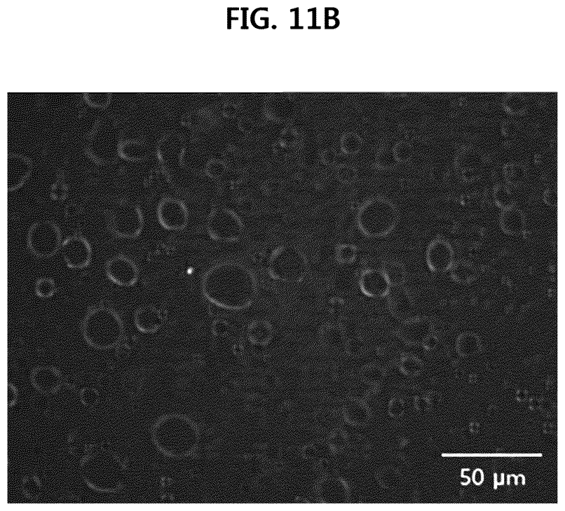

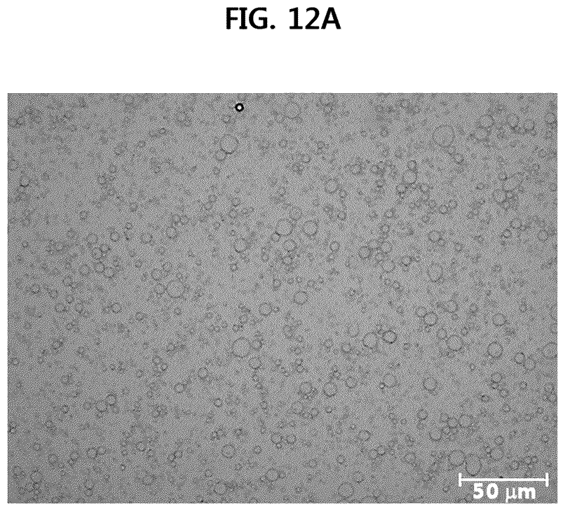

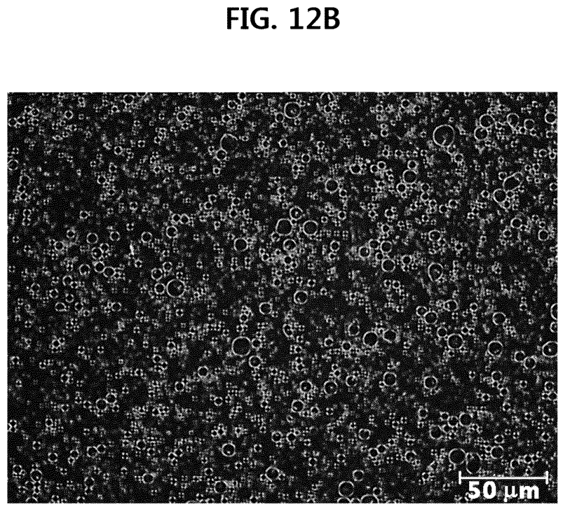

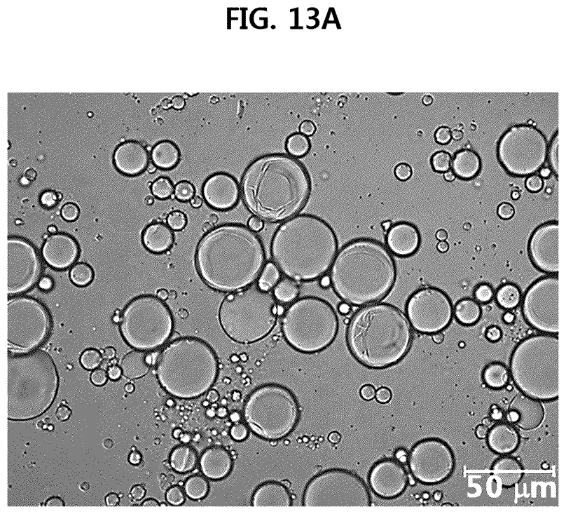

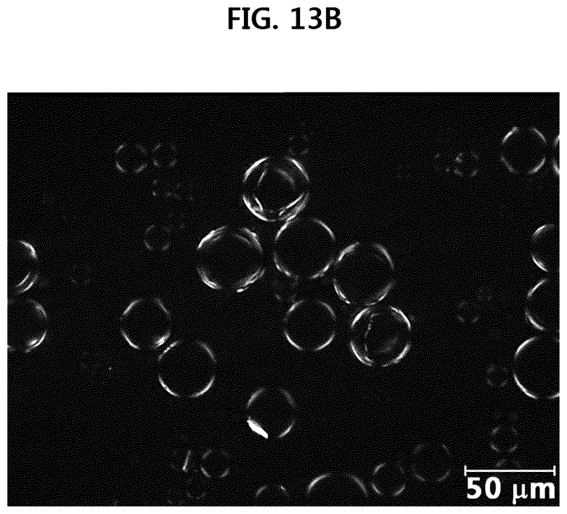

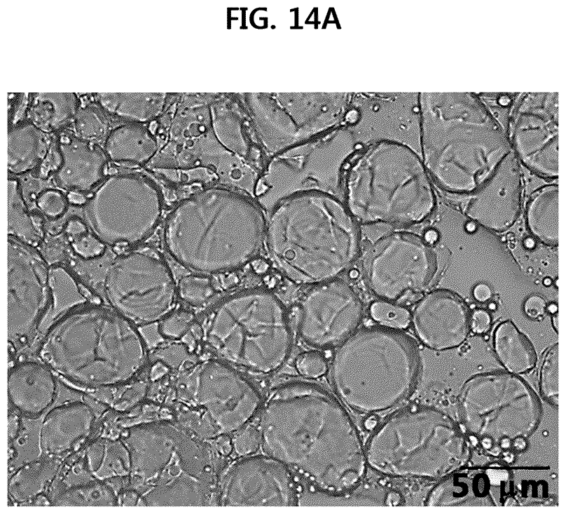

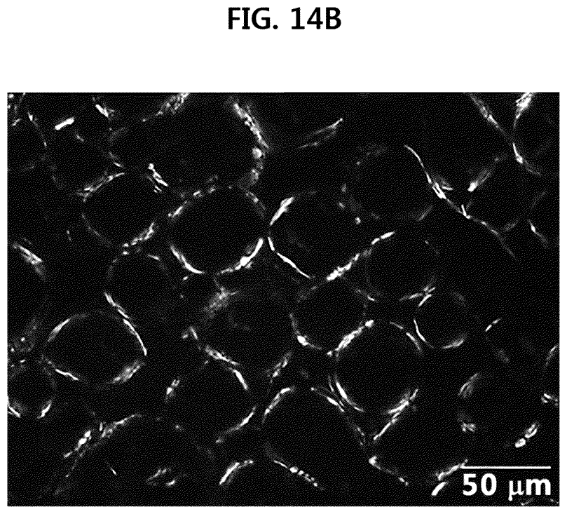

As a result, Example 4 comprising less than 3% by weight of squalane, as shown in FIGS. 11A and 11B, showed a stable emulsified state due to the formation of gel particles. In Example 5, in which the amount of squalane was increased to 3% by weight or more, the oil could not be stably supported at 45° C. for a long period of more than one month due to the limit of the gel particle size. As shown in FIGS. 12A and 12B, in Example 6 comprising lauryl alcohol having 12 carbon atoms, the gel particles had an average size of 1 μm to 10 μm or less. As shown in FIGS. 13A and 13B, Example 7 comprising stearyl alcohol having 18 carbon atoms produced gel particles with an average size of 30 to 100 μm, and showed a stable emulsified state even though 5% by weight of oil was comprised. As shown in FIGS. 14A and 14B, in Example 8 comprising behenyl alcohol having 22 carbon atoms, large gel particles with an average size of 100 μm or more and small gel particles with an average size of 30 μm or less coexisted. This means that in the case that lipids are further comprised in the inner film, the size of the gel particles can be adjusted according to the number of carbon atoms in the lipids. In addition, in the case of Example 7 comprising lipids having 13 to 21 carbon atoms, it can be confirmed that oil can be stably comprised at a high content of 3% by weight or more as an active substance.

### Experimental Example

In order to confirm the skin absorbability of the composition according to the disclosure, the following experiment was conducted.

In one embodiment of the disclosure, a composition (Example 7) in which 0.1% by weight of saponin (product name: BioGf1K manufacturer: HYUNDAI BIOLAND Co., Ltd) and 5% by weight of squalane were comprised based on the total weight of the composition as the active substances was prepared in the same manner as in Example 1.

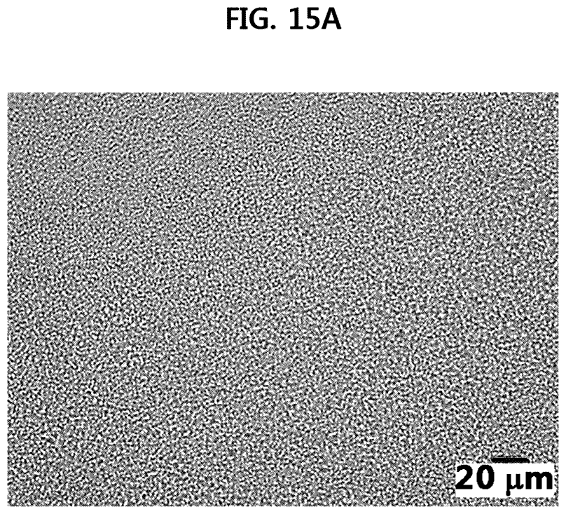

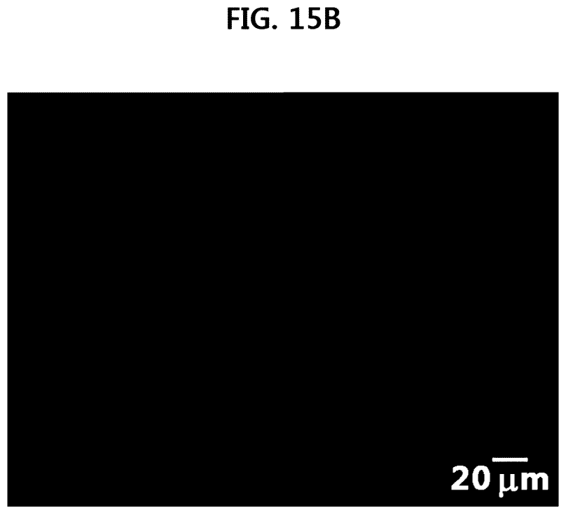

As a comparative example, an O/W emulsion type composition (Comparative Example 5) comprising the composition of Table 3 below and 0.1% by weight of saponin (product name: BioGf1K, manufacturer: HYUNDAI BIOLAND Co., Ltd) based on the total weight of the composition as the active substance was prepared according to a method commonly employed in the art. As another comparative example, Comparative Example 6 was prepared by dissolving 0.1% by weight of saponin (product name: BioGf1K, manufacturer: HYUNDAI BIOLAND Co., Ltd) in polyol and oil part. FIGS. 15A and 15B show the formulation of Comparative Example 5 immediately after preparation Comparative Example 5 with an optical microscope and a polarizing microscope, respectively, and the polarizing thin film was not observed.

TABLE 3

| | | Comparative |
|---|---|---|
| | INCI name | Example 5 |
| Aqueous solvent | Water | To 100 |
| Alcohol solvent | Glycerin | 5 |
| | 1,2-hexanediol | 1 |
| | Lauryl alcohol | — |
| | Behenyl alcohol | — |
| | Stearyl alcohol | 0.5 |
| Active substance | Squalane | 5 |
| Amphiphilic natural | Zein | — |
| protein stabilizer | | |
| Pullulan | Pullulan | — |
| Active substance | Hydrolyzed ginseng | 0.1 |
| | saponin, BioGF1K | |
| Synthetic emulsifier | PEG stearate | 1 |
| Anionic natural | Pectin | — |
| polymer | | |
| Gelling agent | Calcium chloride | — |
| Thickener | Hydroxyethyl cellulose | 5 |
| Total (wt %) | 100 | 100 |

In Example 7, the skin absorption rate of the active substance, saponin was tested using a Franz diffusion cell system (model name: Franz diffusion cells manufacturer: Teledyne), and the test conditions were 10% EtOH receptor, 300 rpm, and 32±0.5° C. Specifically, Example 7, Comparative Example 5, and Comparative Example 6 were loaded in a Franz cell at 0.5 g (biogf1K 0.1% ), and after 24 hours, the concentration detected on the Strat-MTM membrane was measured using high performance liquid chromatography(HPLC) and confirmed. Concentration above the limit of quantification(LOQ) was detected in all experimental groups. The Strat-M™ membrane used in this experiment is composed of a multi-layer that shows various dissolution rates like human skin, that is, two layers of polyethersulfone(PES) and a mixture layer of polyolefin (porous structure), so it has the same characteristics as real skin. Therefore, it shows strong similarity and reproducible results with human skin in various compounds.

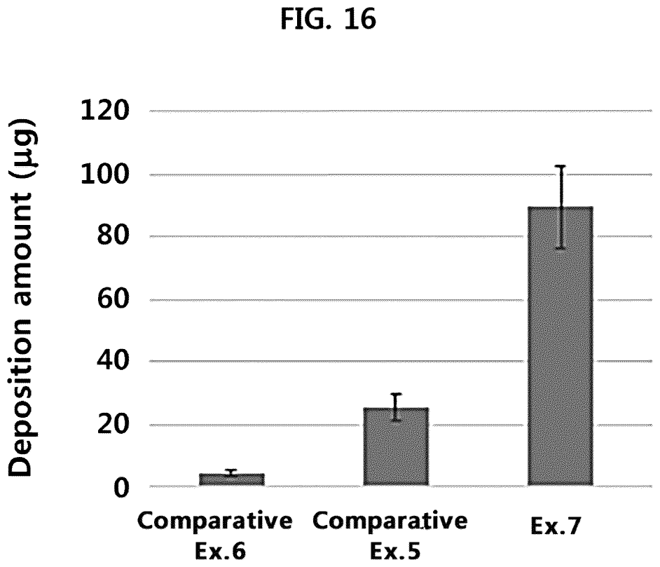

FIG. 16 is a diagram showing measurement and comparison of skin absorbability of Example 7 according to one embodiment of the disclosure and Comparative Examples 5 and 6. As shown in FIG. 16, Comparative Example 6 in which the same concentration of saponin was dispersed in purified water showed a very low absorption rate, whereas Example 7, which is an example of the disclosure, showed a high absorption rate with an efficiency of 89.35% This is a skin absorption rate more than three times higher than the absorption rate of 25.56% of Comparative Example 5, which is the general emulsifier type.

The disclosure may provide the following embodiments as one embodiment.

### First Embodiment

An emulsion composition comprising a gel particle comprising an inner film comprising pullulan and an outer jelly coat film comprising an anionic natural polymer and a gelling agent; and an active substance and an amphiphilic natural protein surfactant in the inner film.

### Second Embodiment

The composition according to first embodiment, wherein the anionic natural polymer is a vegetable polysaccharide.

### Third Embodiment

The composition according to first or second embodiment, wherein the anionic natural polymer is pectin, alginic acid, hyaluronic acid, starch, dextran, carrageenan, cellulose, agarose, agar or a combination thereof, or a salt thereof.

### Fourth Embodiment

The composition according to any one of first to third embodiments, wherein the gelling agent comprises one or more selected from the group consisting of calcium chloride, calcium carbonate, calcium oxide and calcium sulfate.

### Fifth Embodiment

The composition according to any one of first to fourth embodiments, wherein the amphiphilic natural protein surfactant comprise one or more selected from the group consisting of zein, hordein, secalin, kafirin, gliadin, oryzin and avenin.

### Sixth Embodiment

The composition according to any one of first to fifth embodiments, wherein an average particle size of the gel particle is 1 to 2000 μm.

### Seventh Embodiment

The composition according to any one of first to sixth embodiments, wherein the inner film further comprises a lipid.

### Eighth Embodiment

The composition according to any one of first to seventh embodiments, wherein the lipid has 6 to 30 carbon atoms in a hydrophobic tail.

### Ninth Embodiment

The composition according to any one of first to eighth embodiments, wherein a size of the gel particle is adjusted according to a number of carbon atoms in the lipid.

### Tenth Embodiment

The composition according to any one of first to ninth embodiments, wherein the active substance comprises a solid hydrophobic active substance, an oil-soluble liquid active substance, or a mixture thereof.

### Eleventh Embodiment

The composition according to any one of first to tenth embodiments, Wherein the oil-soluble liquid active substance comprises oil.

### Twelfth Embodiment

The composition according to any one of first and eleventh embodiments, wherein the composition comprises 0.1 to 10% by weight of the active substance based on a total weight of the composition.

### Thirteenth Embodiment

The composition according to any one of first to twelfth embodiments, wherein the composition is a cosmetic composition.

### Fourteenth Embodiment

The composition according to any one of first to thirteenth embodiments, Wherein the composition is a pharmaceutical composition.

### DESCRIPTION OF REFERENCE NUMERALS

1: active substance
2: amphiphilic natural protein stabilizer
3: inner film
4: outer jelly coat film
5: lipid

What is claimed is:

1. An emulsion composition comprising:
a gel particle comprising:
an inner film comprising pullulan; and
an outer jelly coat film comprising an anionic natural polymer and a gelling agent; and
an active substance and an amphiphilic natural protein surfactant in the inner film.

2. The composition of claim 1, wherein the anionic natural polymer is a vegetable polysaccharide.

3. The composition of claim 1, wherein the anionic natural polymer is pectin, alginic acid, hyaluronic acid, starch, dextran, carrageenan, cellulose, agarose, agar or a combination thereof, or a salt thereof.

4. The composition of claim 1, wherein the gelling agent comprises one or more selected from the group consisting of calcium chloride, calcium carbonate, calcium oxide and calcium sulfate.

5. The composition of claim 1, wherein the amphiphilic natural protein surfactant comprise one or more selected from the group consisting of zein, hordein, secalin, kafirin, gliadin, oryzin and avenin.

6. The composition of claim 1, wherein an average particle size of the gel particle is 1 to 2000 μm.

7. The composition of claim 1, wherein the inner film further comprises a lipid.

8. The composition of claim 7, wherein the lipid has 6 to 30 carbon atoms in a hydrophobic tail.

9. The composition of claim 8, wherein a size of the gel particle is adjusted according to a number of carbon atoms in the lipid.

10. The composition of claim 1, wherein the active substance comprises a solid hydrophobic active substance, an oil-soluble liquid active substance, or a mixture thereof.

11. The composition of claim 10, wherein the oil-soluble liquid active substance comprises oil.

12. The composition of claim 1, wherein the composition comprises 0.1% to 10% by weight of the active substance based on a total weight of the composition.

13. The composition of claim 1, wherein the composition is a cosmetic composition.

14. The composition of claim 1, wherein the composition is a pharmaceutical composition.
